SEQUENCE LISTING (2 sequences)

SEQ ID NO: 1 - DNA, 19 bp, Homo sapiens
catcattaccaggcatatt

SEQ ID NO: 2 - RNA, 19 nt, Homo sapiens
caucauuaccaggcauauu

(19) United States
(12) Patent Application Publication
(10) Pub. No.: US 2022/0333107 A1
(43) Pub. Date: Oct. 20, 2022

(54) VASCULOGENIC FIBROBLASTS

(71) Applicant: THE TRUSTEES OF INDIANA UNIVERSITY, Bloomington, IN (US)

(72) Inventors: Chandan K. SEN, Indianapolis, IN (US); Kanhaiya SINGH, Indianapolis, IN (US); Sashwati ROY, Indianapolis, IN (US); Mervin C. YODER, Indinaapolis, IN (US); Saba TABASUM, Indianapolis, IN (US); Ahmed Safwat ABOU-HASHEM, Indiaapolis, IN (US); Subhadip GHATAK, Indianapolis, IN (US)

(21) Appl. No.: 17/639,263

(22) PCT Filed: Sep. 18, 2020
(86) PCT No.: PCT/US2020/051603

§ 371 Date: Feb. 28, 2022

Related U.S. Application Data
(60) Provisional application No. 62/903,130, filed on Sep. 20, 2019.
(60) Provisional application No. 62/906,140, filed on Sep. 26, 2019.

(51) Int. Cl. C12N 15/113; C12N 5/077; A61P 9/00

(52) U.S. Cl. CPC C12N 15/113; C12N 5/0656; A61P 9/00; C12N 2310/14; C12N 2320/30

(57) ABSTRACT

Compositions and methods are provided for reprogramming dermal fibroblasts to exhibit vasculogenic properties including the ability to stimulate vasculogenesis in vivo. In accordance with one embodiment such compositions are used in conjunction with standard treatment for use on chronic wounds including in diabetic patients.

### CROSS REFERENCE TO RELATED APPLICATIONS

This application claims priority to the following: U.S. Provisional Patent Application No. 62/903,130 filed on Sep. 20, 2019 and U.S. Provisional Patent Application No. 62/906,140 filed on Sep. 26, 2019, the disclosure of which are expressly incorporated herein.

### STATEMENT OF US GOVERNMENT SUPPORT

This invention was made with government support under GM108014 awarded by the National Institutes of Health. The government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 1 kilobytes ACII (Text) file named “322025_ST25.txt,” created on Sep. 18, 2020.

### BACKGROUND OF THE DISCLOSURE

Fibroblasts are the most common cells of connective tissue in animals. Unlike the epithelial cells lining the body structures, fibroblasts do not form flat monolayers and are not restricted by a polarizing attachment to a basal lamina on one side. The main function of fibroblasts is to maintain the structural integrity of connective tissues by continuously secreting precursors of the extracellular matrix. Fibroblasts secrete the precursors of all the components of the extracellular matrix, primarily the ground substance and a variety of fibers.

Significant fibroblast heterogeneity has recently been described in human and murine tissues. This in combination with further understandings of how fibroblast behavioral state changes are necessary to understand tissue development and homeostasis, provide new strategies for tissue regeneration and addressing disease states relating to inappropriate or undesirable fibroblast activity.

Descriptions of distinct fibroblast subpopulations that display specific behavior state changes during development or in response to injury are only now emerging. As disclosed herein applicant has combined single-cell RNA sequencing with detailed lineage specific fibroblast functional and molecular analysis to identify novel fibroblast subpopulation state changes that are physiologically epigenetically regulated follow acute injury.

### SUMMARY

In accordance with one embodiment of the present disclosure, compositions and methods are provided for reprogramming human dermal fibroblasts to be vasculogenic wherein the reprogrammed dermal fibroblasts have the capacity to induce the formation of blood vessels. The method can be conducted on dermal fibroblasts in vivo or in vitro. In one embodiment the method comprises reducing the miR-200b abundance within fibroblasts to produce cells retaining fibroblast characteristics including for example the presence of fibroblast specific protein-1 (Fsp-1) while exhibiting vasculogenic properties, including for example the ability to form lumenized capillary like structures in culture and/or in vivo. In one embodiment anti-miR-200b oligonucleotides (e.g. interference RNAs) are transfected into human dermal fibroblasts (HADF) expressing fibroblast specific protein-1 (Fsp-1) to modify the HADF to produce vasculogenic fibroblasts that exhibit vasculogenic properties. In one embodiment the vasculogenic properties include one or more of the following properties:

endothelial cell (EC)-like morphologic shape changes;

upregulation of cell surface vascular endothelial growth factor receptor-2 (VEGFR2) expression;

upregulated expression of platelet endothelial cell adhesion molecule 1 (PECAM-1), also known as cluster of differentiation 31 (CD31);

upregulation of endothelial nitric oxide synthase (eNOS);

upregulation of cadherin 5 (CDH5), and

enhanced uptake of acetylated low density lipoprotein (Ac-LDL). In addition the vasculogenic fibroblasts disclosed herein exhibit the ability to form tubular structures upon plating on Matrigel, and/or lumenized structures in 3 dimensional (D) type 1 collagen gels, and/or formed chimeric lumenized capillary like structures in 3D gel co-cultures with cord blood endothelial colony forming cells. In one embodiment a vasculogenic fibroblast is provided wherein the vasculogenic fibroblast one or more proteins uregulated or down regulated as indicated in FIG. 2B relative to native dermal fibroblast that have not been reprogrammed as described in the present application.

In one embodiment a vasculogenic fibroblast cell is provided, wherein the cell expresses both fibroblast specific protein-1 (Fsp-1) and vascular endothelial growth factor receptor-2 (VEGFR2). Optionally the cell may express additional markers of endothelial cells selected from the group consisting of platelet endothelial cell adhesion molecule 1 (CD31), endothelial nitric oxide synthase (eNOS), and cadherin 5 (CDH5). The vasculogenic fibroblasts disclosed herein can be induced to assist in stimulating vascularization at a desired site in a patient.

### BRIEF DESCRIPTION OF THE DRAWINGS

FIGS. 1A and 1B: Direct reprogramming of dermal fibroblasts to vasculogenic state by an anti-miR-200b oligonucleotide. Locked nucleic acid (LNA) targeting miR-200b: 5′-catcattaccaggcatatt-3′ (SEQ ID NO: 1) with phosphorothioate modifications was used. Successful delivery of this 19-mer ASO was verified by RT-qPCR using custom primers. A similar 19-mer oligonucleotide having no hits of >70% homology to any sequence in any organism in the NCBI and miRBase databases was used as sham control Immunocytochemistry of FSP-1/CD-31 colocalization of human adult dermal fibroblast (HADF) cells transfected with control or miR-200b inhibitor were observed. Cells were counter stained with DAPI. (n=3; results not shown); FIG. 1A Quantification of FSP1 and CD31 in control or miR-200b inhibitor transfected HADF cells on day 7. FIG. 1B Images of triple positive FSP1+VEGFR2+CDH5+ fibroblast at d7 post miR-200b inhibition were observed and a graph of the calculated APC intensity is provided.

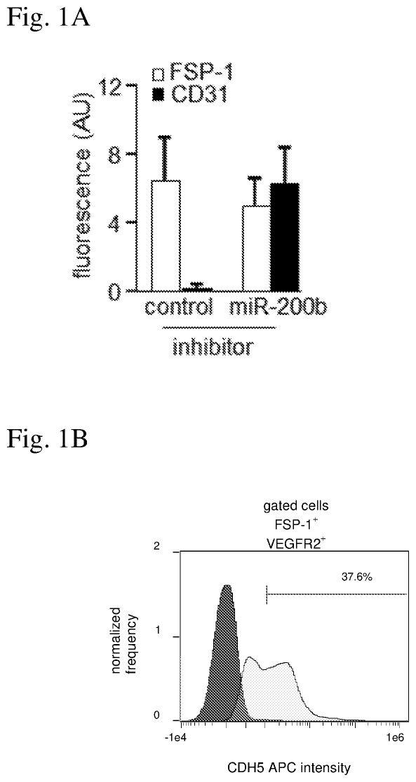

FIGS. 2A and 2B: Single-cell RNA-seq analysis identify novel fibroblast subpopulation state change. Uniform Manifold Approximation and Projection (UMAP) plots showing single-cell transcriptomes of 12880 FSP+VEGFR2-fibroblasts and 11333 FSP+VEGFR2+ fibroblasts were analyzed using 10× Genomics platform. Unsupervised clustering identified 13 clusters. FIG. 2A provides a bar graph showing number of cells in each cluster for fibroblasts (FSP+VEGFR2−) and vasculogenic fibroblasts (FSP+VEGFR2+). Single-cell analysis revealed gain of angiogenic features in certain clusters of vasculogenic fibroblasts post miR-200b inhibition. High levels of pro-angiogenic markers CITED2, GLUL, RRAS and PDGFRB and low levels of anti-angiogenic markers SERPINE1, PGK1, PDCD10 and ITGB1BP1 were detected in FSP+VEGFR2+ fibroblasts. Other signature genes that increased day wise post miR-200b inhibition were: VEGFB, VEGFC, NRP1, NRP2, FLT1, VEGFR1, VEGFR2, MAP2K1, MAP2K2, RAF1, KRAS, NOS3, and PTEN. Molecular characteristics of vasculogenic fibroblasts are provided in FIG. 2B.

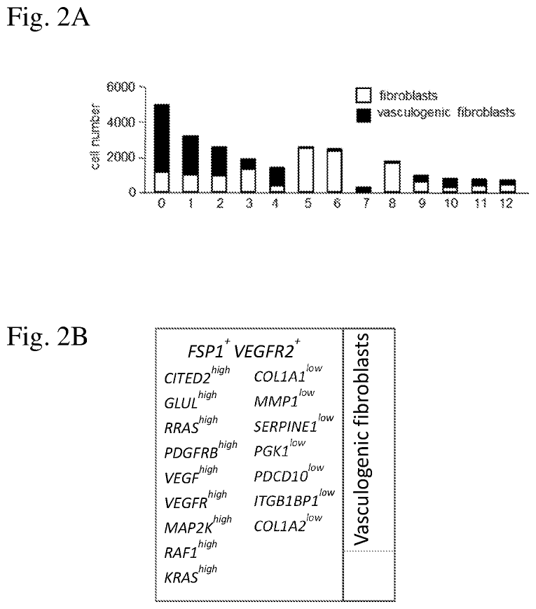

FIGS. 3A and 3B: Increased ischemic tissue perfusion by miR-200b inhibition. FIG. 3A presents pictures of laser speckle imaging (LSI) perfusion data (top), ultrasound (middle) and blood flow velocity (bottom) of sham treated (left panel) and miR-200b treated hind limb ischemia tissue in C57BL/6 mice at d14 post-ischemia (right panel). FIG. 3B is a graph presenting quantitation of LSI perfusion data wherein the dashed line represents LNA control and the solid line represents the LNA miRNA-200b. Results represents the LAN anti-miRNA-200b treated mice (mean±SEM. (n=11)) demonstrating increased perfusion in anti-miRNA-200b treated mice.

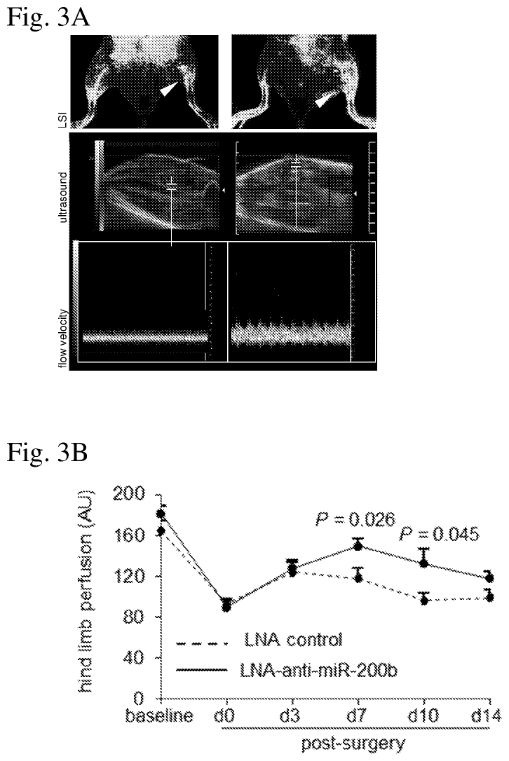

FIGS. 4A and 4B: Increased diabetic wound tissue perfusion by miR-200b inhibition. FIG. 4A presents cutaneous blood perfusion quantification at day 0 and up to day 10 wound-edge tissue of db/db mice treated with either LNA-control or miR-200b inhibitor. (n=4). FIG. 4B presents digital wound planimetry showing more wound contraction at day-8 and day-10 in diabetic wound tissue post-miR-200b inhibition (solid line) relative to control inhibitor (dashed line).

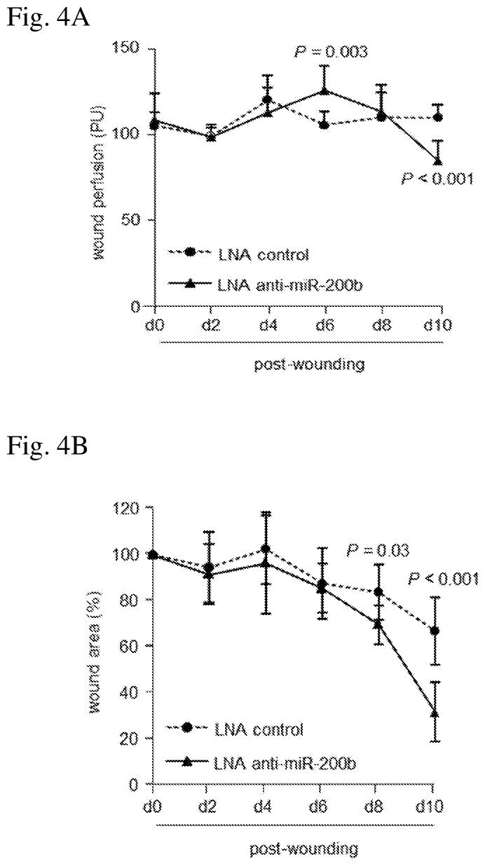

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine (hCys)

V. Large, aromatic residues:
Phe, Tyr, Trp, acetyl phenylalanine, napthylalanine (Nal)

As used herein the term “patient” without further designation is intended to encompass any warm blooded vertebrate domesticated animal (including for example, but not limited to livestock, horses, cats, dogs and other pets) and humans.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein “Interfering RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

As used herein a “locked nucleic acid” (LNA), is a modified RNA nucleotide in which the ribose moiety is modified with an extra bridge connecting the 2′ oxygen and 4′ carbon. For example, a locked nucleic acid sequence comprises a nucleotide of the structure:

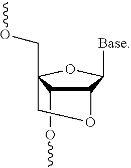

As used herein the term “vasculogenesis” is defined as the differentiation of precursor cells (angioblasts) into endothelial cells and the de novo formation of a primitive vascular network.

### EMBODIMENTS

The present disclosure is directed to a vasculogenic fibroblast that is capable of inducing vasculogenesis in vivo. The vasculogenic fibroblasts disclosed herein are derived from validated human dermal fibroblasts (HADF) expressing fibroblast specific protein-1 (Fsp-1) that have been manipulated to have the capacity to induce the formation of blood vessels. Such reprogrammed fibroblasts exhibit upregulation of cell surface vascular endothelial growth factor receptor-2 (VEGFR2) expression (and other endothelial cell associated proteins and exhibit other molecular signatures of endothelial cells) as well as continued expression of fibroblast specific protein-1 (Fsp-1). These FSP-1+/VEGFR2+ cells surprisingly continued to remodel and contract the collagen gels as efficiently as HADF cells; control human microvascular endothelial cells (HMEC) displayed no such activity. Accordingly, the present disclosure is directed to fibroblast cells that have been induced to have vasculogenic properties.

Applicant has observed that miR-200b abundance is transiently diminished for the first 7 days following full thickness skin injury and as disclosed herein this loss is restricted to dermal fibroblasts present in the wound edge and not in adjacent keratinocytes or tissue macrophages. To assess the biologic significance of this finding, in vitro application of (ASO) to validated human dermal fibroblasts (HADF) expressing fibroblast specific protein-1 (Fsp-1) resulted in the stimulation of >25% of the ASO transfected cells to undergo endothelial cell (EC)-like morphologic shape changes and upregulation of cell surface vascular endothelial growth factor receptor-2 (VEGFR2) expression. FSP-1+/VEGFR2+ cells also upregulated expression of platelet endothelial cell adhesion molecule 1 (CD31), endothelial nitric oxide synthase (eNOS), cadherin 5 (CDH5), and enhanced uptake of acetylated low density lipoprotein (Ac-LDL); normal features of endothelial cells (ECs), but not seen in normal fibroblast cells. FSP-1+/VEGFR2+ cells also formed tubular structures upon plating on Matrigel, lumenized structures in 3 dimensional (D) type 1 collagen gels, and formed chimeric lumenized capillary like structures in 3D gel co-cultures with cord blood endothelial colony forming cells; all EC-like behaviors.

In accordance with one embodiment a method is provided for reprogramming human dermal fibroblast cells to exhibit one or more vasculogenic properties relative to the original fibroblast cell, wherein the method comprises decreasing the concentration of functional miR-200b in said cells. As used herein decreasing the concentration of functional miRNA-200b includes an actual decrease in intracellular miRNA-200b concentrations, and/or a decrease in the percentage of miRNA-200b present in a cell that is capable of conducting native miRNA-200b functions such as regulation of gene expression. In one embodiment the method comprises an actual reduction in detectable miR-200b in the targeted fdermal fibroblasts. In one embodiment the reprogrammed fibroblasts continue to express fibroblast specific protein-1 (Fsp-1), and exhibit at least of the following properties: endothelial cell (EC)-like morphologic shape changes; upregulation of cell surface vascular endothelial growth factor receptor-2 (VEGFR2) expression; upregulated expression of platelet endothelial cell adhesion molecule 1 (CD31); upregulation of endothelial nitric oxide synthase (eNOS); upregulation of cadherin 5 (CDH5), and enhanced uptake of acetylated low density lipoprotein (Ac-LDL).

In accordance with one embodiment a method is provided for reprogramming human dermal fibroblast cells to exhibit one or more vasculogenic properties relative to the original fibroblast cell, wherein the method comprises delivering intracellularly into the fibroblasts one or more proteins selected from the group consisting of ETV2, FOXC2, and FLI1, or polynucleotides encoding one or more proteins selected from the group consisting of ETV2, FOXC2, and FLI1 proteins; or exposing the fibroblasts to an extracellular vesicle produced from a cell containing or expressing one or more proteins selected from the group consisting of ETV2, FOXC2, and FLI1, or polynucleotides encoding one or more proteins selected from the group consisting of ETV2, FOXC2, and FLI1 proteins.

In one embodiment the method of reprogramming the fibroblasts comprises decreasing intracellular miR-200b concentrations, wherein the miR-200b concentration is decreased by transfecting cells with an interference oligonucleotide. The transfection can take place in vivo or in vitro. In one embodiment an anti-miR-200b oligonucleotide is delivered into the cytosol of human dermal fibroblast cells, optionally wherein the anti-miR-200b oligonucleotide is delivered into the cytosol of human dermal fibroblast cells in vivo. In one embodiment the anti-miR-200b oligonucleotide comprises the sequence of SEQ ID NO: 1 or an RNA counterpart thereof.

In accordance with the present invention nucleic acid and/or proteins are introduced into the cytosol of dermal fibroblasts to induce reprogramming of the target cells. Any of the standard techniques for introducing macromolecules into cells can be used in accordance with the present invention. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload.

In one embodiment intracellular delivery is via a viral vector, or other delivery vehicle capable of interacting with a cell membrane to deliver its contents into a cell. In one embodiment intracellular delivery is via three-dimensional nanochannel electroporation, delivery by a tissue nanotransfection device, or delivery by a deep-topical tissue nanoelectroinjection device. In one embodiment the reprogramming composition is delivered into the cytosol of fibroblasts in vivo through tissue nanotransfection (TNT) using a silicon hollow needle array.

Among the methods of permeabilization-based disruption delivery, electroporation has already been established as a universal tool. High efficiency delivery can be achieved with minimum cell toxicity by careful control of the electric field distribution. In accordance with one embodiment nucleic acid sequences are delivered to the cytosol of somatic cells through the use of tissue nanotransfection (TNT). Tissue nanotransfection (TNT) is an electromotive gene transfer technology that delivers plasmids, RNA and oligonucleotides to live tissue causing direct conversion of tissue function in vivo under immune surveillance without the need for any laboratory procedures. Unlike viral gene transfer commonly used for in vivo tissue reprogramming, TNT obviates the need for a viral vector and thus minimizes the risk of genomic integration or cell transformation.

Current methods of in vivo reprogramming can involve transfecting cells in vivo or in vitro followed by implantation. Although one embodiment of the present invention entails in vitro reprogramming of cells followed by transplantation, cell implants are often met with low survival and poor tissue integration. Additionally, transfecting cells in vitro involves additional regulatory and laboratory hurdles.

In accordance with one embodiment dermal fibroblasts are transfected in vivo with a reprogramming composition as disclosed herein. Common methods for bulk in vivo transfection are delivery of viral vectors or electroporation. Although viral vectors can be used in accordance with the present disclosure for delivery of a reprogramming composition to dermal fibroblasts, viral vectors suffer the drawback of potentially initiating undesired immune reactions. In addition, many viral vectors cause long term expression of gene, which is useful for some applications of gene therapy, but for applications where sustained gene expression is unnecessary or even undesired, transient transfection is a viable option. Viral vectors also involve insertional mutagenesis and genomic integration that can have undesired side effects. However, in accordance with one embodiment certain non-viral carriers, such as liposomes or exosomes can be used to deliver a reprogramming cocktail to somatic cells in vivo.

TNT provides a method for localized gene delivery that causes direct conversion of tissue function in vivo under immune surveillance without the need for any laboratory procedures. By using TNT with plasmids, it is possible to temporally and spatially control overexpression of a gene or inhibit expression of a target gene. Spatial control with TNT allows for transfection of a target area such as a portion of skin tissue without transfection of other tissues. Details regarding TNT devices have been described in US published patent application nos. 20190329014 and 20200115425, the disclosures of which are expressly incorporated by reference. Tissue nanotransfection allows for direct cytosolic delivery of cargo (e.g, reprogramming factors) into cells by applying a highly intense and focused electric field through arrayed nanochannels, which benignly nanoporates the juxtaposing tissue cell members, and electrophoretically drives cargo into the cells.

In accordance with one embodiment a vasculogenic fibroblast produced by any one of the methods disclosed herein is provided wherein the vasculogenic fibroblast expresses fibroblast specific protein-1 (Fsp-1), and at least one protein selected from the group consisting of vascular endothelial growth factor receptor-2 (VEGFR2) expression, platelet endothelial cell adhesion molecule 1 (CD31), endothelial nitric oxide synthase (eNOS) and cadherin 5 (CDH5). In one embodiment the vasculogenic fibroblast is characterized by elevated expression of one or more of CITED2, GLUL, RRAS, PDGFRB, VEGF, VEGFR, MAP2K, RAF1, KRAS, optionally by elevated expression of all of CITED2, GLUL, RRAS, PDGFRB, VEGF, VEGFR, MAP2K, RAF1, KRAS and/or low expression of one or more of COL1A1, MMP1, SERPINE1, PGK1, PDCD10, ITGB1BP1 and COL1A2, optionally by low expression of all of COL1A1, MMP1, SERPINE1, PGK1, PDCD10, ITGB1BP1 and COL1A2.

In one embodiment the vasculogenic fibroblast is provided wherein the fibroblast expresses fibroblast specific protein-1 (Fsp-1), and at least one protein selected from the group consisting of vascular endothelial growth factor receptor-2 (VEGFR2) expression, platelet endothelial cell adhesion molecule 1 (CD31), endothelial nitric oxide synthase (eNOS) and cadherin 5 (CDH5). Optionally the vasculogenic fibroblast of the present disclosure further comprises an interference RNA that targets miRNA-200b and/or an exogenously introduce nucleic acid encoding for one or more of ETV2, FOXC2, and FLI1. In one embodiment the vasculogenic fibroblast is in an isolated or purified state. In one embodiment the vasculogenic fibroblast also exhibits endothelial cell (EC)-like morphologic shape and has enhanced uptake of acetylated low density lipoprotein (Ac-LDL) relative to native dermal fibroblasts. In one embodiment the vasculogenic fibroblast expresses each of said vascular endothelial growth factor receptor-2 (VEGFR2) expression, platelet endothelial cell adhesion molecule 1 (CD31), endothelial nitric oxide synthase (eNOS) and cadherin 5 (CDH5).

In accordance with the present invention any of the vasculogenic fibroblasts disclosed herein can be used to stimulate neovascularization in a patients tissues, said method comprising the step of reprogramming dermal fibroblasts in vivo to become vasculogenic, or introducing vasculogenic fibroblasts that have been reprogrammed in vitro to be vasculogenic. In one embodiment the dermal fibroblasts have been reprogrammed by contacting said dermal fibroblasts with an anti-miRNA-200b oligonucleotide and/or nucleic acid sequences encoding ETV2, FOXC2, and/or FLI1 under conditions that enhance cellular uptake of said nucleic acid sequences. In one embodiment the reprogramming comprises delivery of an anti-miR-200b oligonucleotide into the cytosol of human dermal fibroblast cells.

In one embodiment a method of enhancing wound repair in diabetic patients is provided wherein the method comprising introducing vasculogenic fibroblasts or reprogramming fibroblasts to become vasculogenic in tissues proximal to said wound. In one embodiment the method comprises transfecting dermal fibroblast with an inhibitor of miR-200b and or enhancing the expression of FLI1 in dermal fibroblasts.

### Example 1

### Identification of a Vasculogenic Fibroblast State

In vitro application of anti-miR-200b oligonucleotides (ASO) to validated human dermal fibroblasts (HADF) expressing fibroblast specific protein-1 (Fsp-1) was determined to stimulate >25% of the ASO transfected cells to undergo endothelial cell (EC)-like morphologic shape changes and upregulation of cell surface vascular endothelial growth factor receptor-2 (VEGFR2) expression. FSP-1+/VEGFR2+ cells also upregulated expression of platelet endothelial cell adhesion molecule 1 (CD31), endothelial nitric oxide synthase (eNOS), cadherin 5 (CDH5), and enhanced uptake of acetylated low density lipoprotein (Ac-LDL); normal features of EC, but not seen in normal fibroblast cells. FSP-1+/VEGFR2+ cells also formed tubular structures upon plating on Matrigel, lumenized structures in 3 dimensional (D) type 1 collagen gels, and formed chimeric lumenized capillary like structures in 3D gel co-cultures with cord blood endothelial colony forming cells; all EC-like behaviors. However, the FSP-1+/VEGFR2+ cells surprisingly continued to remodel and contract the collagen gels as efficiently as HADF cells; control human microvascular endothelial cells (HMEC) displayed no such activity.

Thus, ASO treatment of HADF diminishes miR-200b abundance and is associated with increases in some EC behaviors, but with retention of classic HADF functions; a novel fibroblast cell state change. To interrogate ASO induced HADF transcriptional changes, single cell RNA sequencing was performed on FSP-1+/VEGFR2+ and FSP-1+/VEGFR2− cells. Unsupervised clustering using the Seurat package identified 13 cell clusters. FSP-1+/VEGFR2+ cells displayed enhanced localization to clusters 0 and 4 with high expression of pro-angiogenic genes and diminished localization to clusters 5, 6, and 8 where numerous fibroblast and anti-angiogenic genes were highly expressed. A ten member gene signature identified the FSP-1+/VEGFR2+ cells in clusters 0 and 4 and the reprogramming trajectory of these enhanced pro-angiogenic cells was identified as a separate branch using pseudotime analysis. These results indicate that ASO treatment of HADF causing a loss of miR-200b abundance resulted in a significant fibroblast subpopulation state change whereby the HADF had become vasculogenic fibroblasts (VF) with gain of EC-like functions.

To identify the molecular regulation of this VF state change, we performed in silico analysis to identify potentially relevant miR-200b target genes. We identified Friend leukemia integration 1 (Fli1), a transcription factor known to be critical for endothelial differentiation and numerous angiogenic responses, as a candidate with miR-200b binding sites in the 3′ untranslated region (3′-UTR). Delivery of a miR-200b mimic significantly suppressed Flit-3′-UTR reporter luciferase activity, but this effect was abrogated in cells with mutated Fli1-3′-UTR. Direct support for the notion that miR-200b targets Fli1 in HADF was obtained from studies using either a miR-200b mimic (diminished FLI1 abundance) or an inhibitor (increased FLI1 abundance). Finally, inhibition of Fli1 transcript abundance markedly blunted the ability of miR-200b inhibition to upregulate FLI1 in VF, as well as, the ensuing vasculogenic gain in function of Matrigel™ tube formation. These observations established that inhibition of miR-200b in HADF in vitro utilizes a molecular pathway common for endothelial differentiation and angiogenesis to induce a VF state change that is FLI1 dependent.

To assess effects of miR-200b inhibition in intact murine skin, LNA anti-miR-200b was applied topically and caused only a transient increase in cutaneous perfusion; demonstrating that miR-200b inhibition alone does not cause a sustained angiogenic outcome in otherwise well-perfused normoxic intact skin. However, under conditions of disrupted cutaneous vasculature, such as in a created wound, miR-200b inhibition at the wound-edge tissue constitutes a physiological response as a component of the healing cascade and leads to increased FLI1 abundance that peaks at the wound edge on day 9. Fibroblast-specific Fli1 transcript abundance was diminished in murine skin by lentivral particle injection of loxP flanked Fli1 shRNA expression cassettes in fibroblast specific Fsp1-Cre:R26RtdTomato transgenic reporter mice and Fli1 knockdown in wound-edge dermal fibroblasts significantly delayed wound perfusion and impaired wound closure. Under conditions of fibroblast-targeted knockdown of FLI1, inhibition of miR-200b failed to bring about a VF cell state transition.

In contrast, when cutaneous wounds were developed on the fibroblast lineage tracing Fsp1-Cre:R26RtdTomato mice and transfected with LNA anti-miR-200b, wound-edge tissue displayed marked abundance of fibroblast lineage marked cells showing VF characteristics. Thus, the miR-200b inhibition that occurs post-wounding and the increased expression of FLI1 in the wound edge are physiologic responses and can be localized to wound edge dermal fibroblasts undergoing a VF state change.

To test the therapeutic significance of miR-200b inhibition, we studied the ischemic hind limb of C57BL6 mice. Tissue nanotransfection (TNT)-based ASO inhibition of miR-200b rescued perfusion and metabolism in the ischemic hindlimb. Micro-CT imaging revealed sprouting of cutaneous microvasculature following miR-200b inhibition. Interestingly, the vasculogenic cells at the site of injury were primarily derived from fibroblasts, and not from other non-endothelial local cells such as keratinocytes and macrophages. Ischemic hind limb studies were also performed on miR-200b-429fl/fl-Col1a2CreER WT and tamoxifen (TAM) treated mice to specifically test the significance miR-200b in skin fibroblasts. Tamoxifen-treated conditional knockout of miR-200b in fibroblasts enhanced perfusion in the ischemic limb and was associated with increased abundance of VF expressing CD31.

To assess whether the miR-200b and Fli1 axis identified in cutaneous wound healing is present in diabetic subjects, we examined wound edge tissue in human patients and observed persistently elevated miR-200b abundance but diminished FLI1 transcripts in diabetic compared to non-diabetic subjects. To test the hypothesis that persistently elevated miR-200b at a wound site alters induction of the VF state and related wound healing, fibroblast specific reporter Fsp1-Cre:R26RtdTomato mice were made diabetic using streptozotocin administration. In these diabetic mice, injury-induced and miR-200b-dependent physiological conversion of fibroblasts to a VF state was impaired.

In db/db mice, another established murine model of type II diabetes, cutaneous wounding failed to suppress miR-200b expression at the wound-edge, similar to human diabetic subjects. ASO-dependent forced miR-200b inhibition at the wound-edge of db/db mice increased the abundance of FLI1 followed by increased abundance of fibroblast to VF cell state change in the wound-edge tissue and was associated with significantly improved wound perfusion and healing.

Thus, we have identified a physiologic role for miR-200b to regulate dermal fibroblast Fli1 expression during wound healing that causes a cell state change in the fibroblasts to a gain in pro-angiogenic VF functional status. This pathway is disrupted in diabetic human and murine subjects, but is remedied via use of TNT delivery of an ASO in diabetic mice to lower the abnormal persistently high miR-200b levels in the injured skin to a point that FLI1 can be upregulated in

expression, the VF state induced, and wound perfusion and healing restored.

This experimental example

of inducing a behavioral state change in fibroblasts, that is now known to occur physiologically in skin, and

can be topically augmented by cutaneous TNT delivery of a single ASO to rescue a pathway that is blunted

in diabetes, suggests new paradigms for considering regenerative therapies.

Rather than consider

approaches of injection or implantation of replacement proteins, cells, or composite tissues, use of topical

approaches that augment known epigenetic pathways may be testable alternatives.

1. A method of reprogramming human dermal fibroblast cells to exhibit one or more vasculogenic properties relative to the original fibroblast cell, wherein the reprogrammed cell exhibits at least one of the following properties:
endothelial cell (EC)-like morphologic shape changes;
upregulation of cell surface vascular endothelial growth factor receptor-2 (VEGFR2) expression;
upregulated expression of platelet endothelial cell adhesion molecule 1 (CD31);
upregulation of endothelial nitric oxide synthase (eNOS);
upregulation of cadherin 5 (CDH5), and
enhanced uptake of acetylated low density lipoprotein (Ac-LDL);
while expressing fibroblast specific protein-1 (Fsp-1), said method comprising decreasing the concentration of functional miR-200b in said cells.

2. The method of claim 1 wherein the miR-200b concentration is decreased by transfecting cells with an interference RNA.

3. The method of claim 1 wherein an anti-miR-200b oligonucleotide is delivered into the cytosol of human dermal fibroblast cells.

4. The method of claim 3 wherein the anti-miR-200b oligonucleotide is delivered into the cytosol of human dermal fibroblast cells in vivo.

5. The method of claim 4 wherein the intracellular delivery is via tissue nanotransfection.

6. A vasculogenic fibroblast produced by the method of claim 5 wherein the vasculogenic fibroblast expresses
fibroblast specific protein-1 (Fsp-1), and
at least one protein selected from the group consisting of vascular endothelial growth factor receptor-2 (VEGFR2) expression, platelet endothelial cell adhesion molecule 1 (CD31), endothelial nitric oxide synthase (eNOS) and cadherin 5 (CDH5).

7. A method of stimulating neovascularization in a patients tissues, said method comprising the step of reprogramming dermal fibroblasts in vivo to become vasculogenic, said method comprising
contacting said dermal fibroblasts with an anti-miRNA-200b oligonucleotide under conditions that enhance cellular uptake of said anti-miRNA-200b oligonucleotide.

8. The method of claim 7 wherein an anti-miR-200b oligonucleotide is delivered into the cytosol of human dermal fibroblast cells.

9. A vasculogenic fibroblast wherein said fibroblast expresses
fibroblast specific protein-1 (Fsp-1), and
at least one protein selected from the group consisting of vascular endothelial growth factor receptor-2 (VEGFR2) expression, platelet endothelial cell adhesion molecule 1 (CD31), endothelial nitric oxide synthase (eNOS) and cadherin 5 (CDH5).

10. The vasculogenic fibroblast of claim 9 further comprises an interference RNA that targets miRNA-200b.

11. The vasculogenic fibroblast of claim 9 wherein the vasculogenic fibroblast also exhibits endothelial cell (EC)-like morphologic shape and has enhanced uptake of acetylated low density lipoprotein (Ac-LDL) relative to native dermal fibroblasts.

12. The vasculogenic fibroblast of claim 9 wherein the vasculogenic fibroblast expresses each of said vascular endothelial growth factor receptor-2 (VEGFR2) expression, platelet endothelial cell adhesion molecule 1 (CD31), endothelial nitric oxide synthase (eNOS) and cadherin 5 (CDH5).
